(19) United States
(12) Plant Patent Application Publication
(10) Pub. No.: US 2003/0014784 P1
(43) Pub. Date: Jan. 16, 2003

(54) Miniature rose plant 'POULarbso'

(76) Inventors: L. Pernille Olesen, Fredensborg (DK); Mogens N. Olesen, Fredensborg (DK)

Correspondence Address: Poulsen Roser Pacific Inc., 2550 Old Military Road, Central Point, OR, 97502, US

(21) Appl. No.: 10/136,452

(22) Filed: Apr. 29, 2002

Related U.S. Application Data
(63) Continuation of application No. 09/607,331, filed on Jun. 30, 2000.

(51) Int. Cl. A01H 5/00

(52) U.S. Cl. PLT/121

(57) ABSTRACT

A new miniature rose plant which has abundant, non-fading, pink flowers and attractive foliage. The variety successfully propagates from softwood cuttings and is suitable for year round production in commercial glasshouses. This new and distinct variety has shown to be uniform and stable in the resulting generations from asexual propagation.

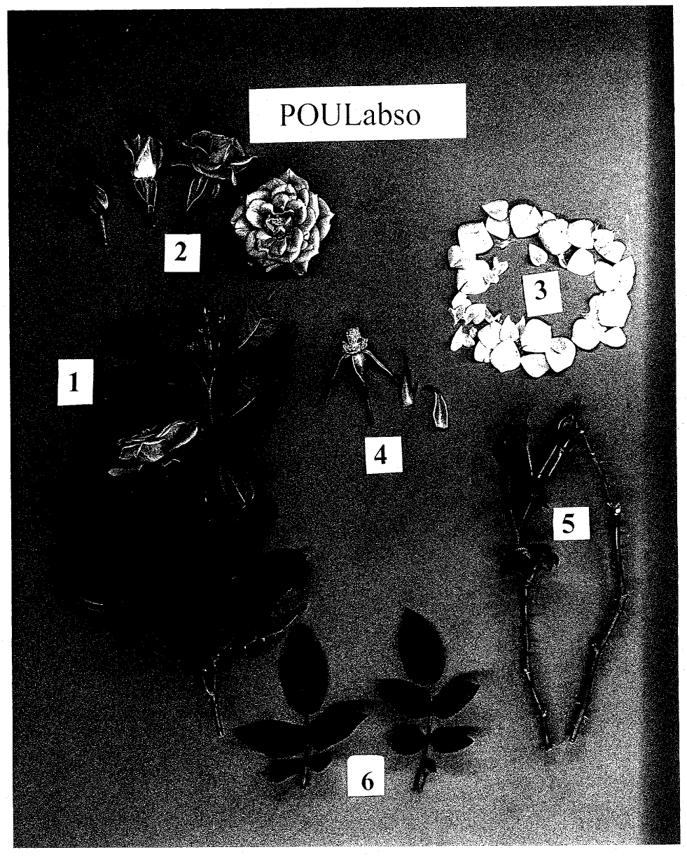

The present invention constitutes a new and distinct variety of miniature rose plant which originated from a controlled crossing between ‘POULrouge’ and an unnamed seedling. The two parents were crossed and the resulting seeds were planted in a controlled environment. The new variety is named ‘POULabso’. The new rose may be distinguished from its seed parent, POULrouge, by the following combination of characteristics:

1. The blooms of ‘POULrouge’ are orange-red, where those of ‘POULabso’ are pink.

2. The flowers of ‘POULrouge’ are flatter than those of ‘POULabso’.

The new variety may be distinguished firom its pollen parent, an unnamed seedling created by the same inventors, by the following combination of characteristics:

1. The unnamed seedling's blooms are dark red, where those of ‘POULabso’ are pink.

2. The growth of the unnamed seedling is closer to the ground than that of ‘POULabso’.

The objective of the hybridization of this rose variety for commercial greenhouse culture was to create a new and distinct variety with unique qualities, such as:

1. Uniform and abundant flowers;

2. Vigorous and compact growth;

3. Year-round flowering under glasshouse conditions;

4. Suitability for production from softwood cuttings in pots;

5. Durable flowers and foliage which make a variety suitable for distribution in the floral industry.

The combination of qualities of this variety represents significant improvement over previously available commercial cultivars of this type and distinguishes ‘POULabso’ from all other varieties of which we are aware.

As part of their rose development program, L. PERNILLE Olesen and Mogens N. Olesen germinated the seeds from the aforementioned hybridization and conducted evaluations on the resulting seedlings in a controlled environment in Fredensborg, Denmark.

‘POULabso’ was selected by the inventors as a single plant from the progeny of the hybridization in Fredensborg, Denmark.

Asexual reproduction of ‘POULabso’ by cuttings and traditional budding was first done by L. PERNILLE and Mogens N. Olesen in Fredensborg, Denmark. This initial and other subsequent propagations conducted in controlled environments have demonstrated that the characteristics of ‘POULabso’ are true to type and are transmitted from one generation to the next.

### BRIEF DESCRIPTION OF THE DRAWING

The accompanying color illustration shows as true as is reasonably possible to obtain in color photographs of this type, the typical characteristics of the buds, flowers, leaves, stems, and a plant of ‘POULabso’. Specifically illustrated in SHEET 1:

1. Stem or entire plant showing branching and the attachment of leaves, buds, and peduncles;

2. Flower bud, partially opened bud, and open bloom;

3. Flower petals, detached;

4. Sepals, receptacle, and pedicel;

5. Flowering stem as well as a bare stem exhibiting thorns;

6. Leaves.

### DETAILED DESCRIPTION OF THE VARIETY

The following is a description of ‘POULabso’, as observed in its growth in glasshouses in Fredensborg, Denmark and Half Moon Bay, Calif. Color references are made using The Royal Horticultural Society (London, England) Colour Chart, 1995, except where common terms of color are used. For a comparison, several physical characteristics of the rose variety ‘POULody’, a minature rose variety from the same inventors described and illustrated in U.S. Plant patent application Ser. No. 09/138,033 dated 08/18/98 are compared to ‘POULabso’ in Chart 1.

CHART 1

| | ’POULabso’ | ’POULody’ |
|---|---|---|
| Petal Count | Very Double; | Very Double; |
| | 40 to 55 Petals. | 50 to 60 Petals. |
| Bud Color at | Red Group 52C | Red-Purple Group |
| ¼ Open. | | 57B-57C |
| Color of Upper | Red Group 48C | Red-Purple Group |
| Surface of Flower | | 57B-57C. |
| Petal On Open | | |
| Flower. | | |

Parents:

Classification:

Botanical.—Rosa hybrida.

Commercial.—Miniature

### FLOWER AND FLOWER BUD

Blooming habit:

Flower bud:

Size.—Upon opening, 19 mm.length from base of receptacle to end of bud.

Bud form.—Long, pointed ovoid.

Bud color.—As sepals unfold, Red Group 52D to Red Group 52C at ¼ opening.

Sepals.—Green Group 143A. Strong foliaceous appendages on three of the five sepals. Surfaces of sepals moderately pubescent. Stipitate glands are present on foliaceous appendages and on the exterior of the sepals.

Receptacle.—Surface: Stipitate glands present. Shape: Funnel. Size: Small; 5 to 6 mm. (h)×2 to 3 mm (w). Color: Yellow-Green Group 143B.

Peduncle.—Surface: Stipitate glands present. Length: 28 to 37 mm. average length. Color: Yellow-Green Group 144A. Strength: Upright.

Borne.—Generally with 2 to 4 buds per flowering stem

Flower bloom:

Fragrance.—Very light; floral.

Duration.—As a pot plant, flowers last from 12 to 15 days. As a cut flower 3 to 4 days

Size.—Medium for a 12 cm. pot rose. Average flower diameter is 45 to 50 mm. when open.

Form.—Shape of flower when viewed from the side: Upon opening, upper part: Flat. Upon opening, lower part: Concave. Open flower, upper part: Concave. Open flower, lower part: Concave.

Petalage.—Very double. Average range: 40 to 55 petals under normal conditions with petaloids.

Color:

Upon opening petals.—Outermost petals: Upper Surface: Red Group 55C. Reverse Side: Red Group 54D. Innermost petals: Upper Surface: Red Group 52D. Reverse Side: Red Group 54C.

Upon opening, basal petal spots.—Outermost petals: Outer Side: Green Yellow Group ID (A little larger on outer petals.). Inner Side: Green Yellow Group ID. Innermost petals: Outer Side: Green Yellow Group 1B. Inner Side: Green Yellow Group 1B. On both sides, the coloration almost forms a line in the center of the petals.

After opening, petals.—Outermost petals: Upper Surface: Red Group 48D. Reverse Side: Red Group 55C. Innermost petals: Upper Surface: Red-Purple Group 58D. Reverse Side: Red-Purple Group 58D.

After opening, basal petal spots.—Outermost petals: Outer Side: Green Yellow Group 1C. Inner Side: Green Yellow Group 1D. Innermost petals: Outer Side: Green Yellow Group 1B. Inner Side: Green Yellow Group 1D. On both sides, coloration describes a stripe up the center of the petal.

General Tonality: On open flower Red Group 52C. No change in the general tonality at the end of the 7th day. Afterwards, general tonality is Red Group 52B.

Petals:

Petal reflex.—Petals reflex somewhat.

Petal edge.—The petals have a point in the center of the margin.

Shape.—Deltoid.

Petaloids.—Present.

Thickness.—Thin.

Arrangement.—Imbricated.

Reproductive Organs:

Pollen.—Color: Yellow-Orange Group 163B. Quantity: Average.

Anthers.—Size: Medium. Color: Greyed Red Group 178B. Quantity: Abundant.

Filaments.—Color: Yellow Group 4A to Yellow Group 4B.

Stigmas.—Stigmas are superior to anthers. Color: Greyed Orange Group 163D.

Styles.—Color: Greyed Orange Group 163D.

### PLANT

Plant growth: Vigorous, upright to bushy. When grown as a 12 cm pot plant, the average height of the plant itself is 20 to 25 cm. andthe average width is 30 to 35 cm.

Stems:

Color.—Young wood: Yellow-Green Group 144A. Older wood: Yellow-Green Group 144A.

Prickles.—Incidence: Moderate. Size: Average length: 4 to 6 mm. Color: Yellow-Green Group 146C. Shape: Deeply concave.

Surface.—Young wood: Smooth. Older wood: Smooth.

Plant foliage: Numbet of leaflets on normal leaves in middle of the stem: 5 leaflets.

Leaf size.—Medium; 39 mm. (l)×25 mm. (w). Terminal leaf is much larger; 40-45 mm. (l)×24-28 mm. (w).

Quantity.—Average to high.

Color.—Upper Leaf Surface: Yellow-Green Group 147A. Lower Leaf Surface: Yellow-Green Group 147B. Juvenile foliage: Green Group 137A. Anthocyanin intonation: Yes. Location: Margins of the stipules, the leaf margin edges on juvenile foliage, young shoots. Color: Greyed-Purple Group 183B to Greyed-Purple Group 183D.

Plant leaves and leaflets:

Stipules.—Size: 7 to 8 mm.(h)×2 to 3 mm (w). Color: Green Group 138A. Presence of stipitate glands: On margins. Pubescence observed on lower surface of stipitates.

Petiole.—Length: 8 to 10 mm. Color: Yellow-Green Group 144B. Underneath: Limited number of stipitate glands and a prickle at stipule attachment and first set of leaves. Margins: Stipitate glands present.

Rachis.—Color: Yellow-Green Group 144B. Underneath: Prickles observed at leaf attachments and occasionally between leaf attachments. Margins: Stipitate glands present.

Leaflet.—Edge: Serrated. Shape: Ovate. Other: Moderately glossy; thin.

Disease resistance: Above average resistance to mildew, black spot, and Botrytis under normal growing conditions in Half Moon Bay, Calif., and Burlington, Canada.

Cold hardiness: ‘POULarbso’ has been found to be resistant to damage from cold, heat and drought damage in USDA Zone 7.

1. A new and distinct variety of rose plant of the floribunda class, substantially as herein illustrated and described as a distinct and novel rose variety due to its abundant, pink flowers, vigorous and upright to growth, year round flowering under glasshouse conditions, suitability for production from softwood cuttings in pots, and durable flowers and foliage which make the variety suitable for distribution in the floral industry:
